(19) United States
(12) Patent Application Publication
(10) Pub. No.: US 2023/0167490 A1
(43) Pub. Date: Jun. 1, 2023

(54) PSEUDO-COMPLEMENTARY BASES IN GENOTYPING AND NUCLEIC ACID SEQUENCING

(71) Applicant: Cepheid, Sunnyvale, CA (US)

(72) Inventors: Russell Higuchi, Alameda, CA (US); Nicholas Wang, Sunnyvale, CA (US)

(73) Assignee: Cepheid, Sunnyvale, CA (US)

(21) Appl. No.: 17/921,582

(22) PCT Filed: Apr. 27, 2021
(86) PCT No.: PCT/US2021/029448

§ 371 Date: Oct. 26, 2022

Related U.S. Application Data
(60) Provisional application No. 63/016,893, filed on Apr. 28, 2020.

(51) Int. Cl. C12Q 1/6855; C12Q 1/6806; C12Q 1/6832; C12Q 1/6876

(52) U.S. Cl. CPC C12Q 1/6855; C12Q 1/6806; C12Q 1/6832; C12Q 1/6876

(57) ABSTRACT

Described herein are methods and compositions that make use of pseudo-complementary bases to reduce unwanted hybridization in assays to detect and/or quantify particular nucleotide sequences, as well as in nucleic acid sequencing protocols.

### CROSS-REFERENCE TO RELATED APPLICATIONS

This application claims the benefit of U.S. provisional application No. 63/016,893, tiled Apr. 28, 2020, which is hereby incorporated by reference in its entirety.

### STATEMENT AS TO RIGHTS TO INVENTIONS MADE UNDER FEDERALLY SPONSORED RESEARCH AND DEVELOPMENT

Not applicable.

### FIELD

The methods and compositions described herein relate generally to the area of detecting or determining nucleotide sequences.

### BACKGROUND

A wide variety of nucleic acid amplification methods are available, and many have been employed in the implementation of sensitive genotyping and diagnostic assays based on nucleic acid detection. Polymerase chain reaction (PCR) remains the most widely used DNA amplification and quantitation method. Nested PCR, a two-stage PCR, is used to increase the specificity and sensitivity of the PCR (U.S. Pat. No. 4,683,195). Nucleic acid amplification is also used in so-called “next-generation” nucleic acid sequencing methods.

Modified DNA bases have been developed that do not base-pair efficiently with one another. Examples are described in U.S. Pat. No. 5,912,340 (issued Jun. 15, 1999 to Kutyavin et al.) and in Woo et al, (1996) “G/C-modified oligodeoxynucleotides with selective complementarity: synthesis and hybridization properties,” Nucleic Acids Research 24(13):2470-2475, both of which are incorporated by reference for this description. These modified bases have been termed “pseudo-complementary” (see, e.g., Lahoud et al. (2008) Nucleic Acids Research 36(10):3409-3419), and pairs of these bases have been referred to as self-avoiding molecular recognition systems (SAMRS; see, e.g., U.S. Pat. No. 8,871,469, issued Oct. 28, 2014 to Benner et al,).

### SUMMARY

Various embodiments contemplated herein may include, but need not be limited to, one or more of the following:

Embodiment 1: A method of determining whether a nucleotide sequence is present in a target nucleic acid sequence in a sample, wherein the target nucleic acid sequence includes a polymorphic site, wherein the polymorphic site is characterized by a first nucleotide sequence and a second nucleotide sequence, wherein the first and second nucleotide sequences differ by at least one nucleotide or ribonucleotide, the method including:

contacting nucleic acid of, or derived from, the sample with forward and reverse primers capable of amplifying the target nucleic acid sequence, wherein said contacting is in the presence of a blocker oligonucleotide that is complementary to the first nucleotide sequence to form a reaction mixture, wherein:
if the target nucleic acid sequence includes the first nucleotide sequence, the blocker oligonucleotide anneals to the first nucleotide sequence and inhibits amplification; or
if the target nucleic acid sequence includes the second nucleotide sequence, the blocker oligonucleotide does not anneal to the second nucleotide sequence and does not inhibit amplification;

conducting an amplification reaction in the reaction mixture;

after the amplification reaction, contacting the reaction mixture, or nucleic acids from the reaction mixture, with a capture oligonucleotide that is complementary to the second nucleotide sequence under conditions suitable for specific hybridization; and

detecting any specific hybridization to the capture oligonucleotide, wherein the presence of specific hybridization to the capture oligonucleotide indicates that the second nucleotide sequence is present in the target nucleic acid sequence, wherein:
the blocker oligonucleotide includes one or more first modified bases and the capture oligonucleotide includes one or more second modified bases, at least one of which is complementary to one of the first modified bases, wherein the modified bases preferentially pair with unmodified forms of their complementary bases, as compared to pairing between modified, complementary bases; and
the presence of the one or more modified bases in the blocker oligonucleotide and in the capture oligonucleotide destabilizes hybridization between the blocker oligonucleotide and the capture oligonucleotide.

Embodiment 2: The method of embodiment 1, wherein at least one of the first modified bases and at least one of the second, complementary modified bases in the capture oligonucleotide are bases that do not differ between the first and second nucleotide sequence.

Embodiment 3: The method of embodiment 1 or embodiment 2, wherein the first nucleotide sequence includes one allele of a gene, and the second nucleotide sequence includes another allele of a gene.

Embodiment 4: The method of embodiment 1 or embodiment 2, wherein the first nucleotide sequence includes a wild-type sequence, and the second nucleotide sequence includes a mutant sequence.

Embodiment 5: The method of any one of embodiments 1 to 4, wherein the polymorphic site is a single nucleotide polymorphism.

Embodiment 6: The method of any one of embodiments 1-5, wherein the amplification includes polymerase chain reaction.

Embodiment 7: The method of any one of embodiments 1-6, wherein the method includes quantifying any specific hybridization to the capture oligonucleotide.

Embodiment 8: The method of any one of embodiments 1-7, wherein the sample consists of nucleic acids from a single cell.

Embodiment 9: An oligonucleotide set including:

forward and reverse primers capable of amplifying a target nucleic acid sequence, wherein the target nucleic acid sequence includes a polymorphic site, wherein the polymorphic site is characterized by a first nucleotide sequence and a second nucleotide sequence, wherein the first and second nucleotide sequences differ by at least one nucleotide or ribonucleotide;

a blocker oligonucleotide that is complementary to the first nucleotide sequence; and

a capture oligonucleotide that is complementary to the second nucleotide sequence, wherein the blocker oligonucleotide includes one or more first modified bases and the capture oligonucleotide includes one or more second modified bases, at least one of which is complementary to one of the first modified bases, wherein the modified bases preferentially pair with unmodified forms of their complementary bases, as compared to pairing between modified, complementary bases; and the presence of the one or more modified bases in the blocker oligonucleotide and in the capture oligonucleotide destabilizes hybridization between the blocker oligonucleotide and the capture oligonucleotide.

Embodiment 10: The method or oligonucleotide set of any one of the preceding embodiments, wherein the capture oligonucleotide is attached to a support.

Embodiment 11: The method or oligonucleotide set of embodiment 10, wherein the support includes a microbead.

Embodiment 12: A method of simplifying preparations for nucleic acid sequencing, the method including:

adding DNA sequencing adaptors to nucleic acid fragments to produce sequencing templates;

amplifying sequencing templates to produce amplified DNA templates; and

contacting the amplified DNA templates with capture oligonucleotides attached to a support under conditions suitable for hybridization, wherein the DNA sequencing adaptors and the capture oligonucleotides comprise complementary nucleotide sequences, wherein:
the DNA sequencing adaptors each comprise one or more first modified bases in their complementary nucleotide sequence, and the capture oligonucleotides comprise one or more second modified bases in their complementary nucleotide sequence, wherein at least one of the first and second modified bases are complementary, wherein the modified bases preferentially pair with unmodified forms of their complementary bases, as compared to pairing between modified, complementary bases; and
hybridization of amplified DNA templates to the capture oligonucleotide is favored over hybridization of free adaptors to the capture oligonucleotides, eliminating a need to separate amplified DNA templates from free adaptors before further DNA sequencing steps.

Embodiment 13: A combination of components for simplifying nucleic acid sequencing, the combination including:

DNA sequencing adaptors;

and capture oligonucleotides attached to, or adapted to be attached to, a support, wherein the DNA sequencing adaptors and the capture oligonucleotides comprise complementary nucleotide sequences, wherein:
the DNA sequencing adaptors each comprise one or more first modified bases in their complementary nucleotide sequence, and the capture oligonucleotides comprise one or more second modified bases in their complementary nucleotide sequence, wherein at least one of the first and second modified bases are complementary, wherein the modified bases preferentially pair with unmodified forms of their complementary bases, as compared to pairing between modified, complementary bases; and
hybridization of amplified DNA templates to the capture oligonucleotide is favored over hybridization of free adaptors to the capture oligonucleotides, eliminating a need to separate amplified DNA templates from free adaptors.

Embodiment 14: The method of embodiment 12 or the combination of components of embodiment 13, wherein the DNA sequencing adaptors comprise a nucleotide sequence that is a binding site for a DNA sequencing primer and a barcode nucleotide sequence.

Embodiment 15: The method of embodiment 12, wherein the method additionally includes producing the nucleic acid fragments from genomic DNA, or the combination of components of embodiment 13, wherein the combination additionally includes one or more reagents that produce the nucleic acid fragments from genomic DNA.

Embodiment 16: The method of embodiment 12, wherein said adding of DNA sequencing adaptors includes ligating the DNA sequencing adaptors to the nucleic acid fragments, or the combination of components of embodiment 13, wherein the combination additionally includes a ligase.

Embodiment 17: The method or combination of components of any one of embodiments 1246, wherein the method employs, or the combination includes, a DNA polymerase for amplification.

Embodiment 18: The method or combination of components of any one of embodiments 12-17, wherein the method employs, or the combination includes, a reverse transcriptase for reverse-transcribing nucleic acid fragment that are RNA.

Embodiment 19: The method or combination of components of any one of embodiments 12-18, wherein the method additionally includes sequencing the amplified DNA templates or the combination additionally includes additional reagents for sequencing DNA.

Embodiment 20: The method, oligonucleotide set, or combination of components of any one of the preceding embodiments, wherein modified complementary bases form fewer hydrogen bonds with each other than with unmodified complementary bases.

Embodiment 21: The method, oligonucleotide set, or combination of components of embodiment 20, wherein the Tₘ of a base pair formed between modified complementary bases less than 40 T.

Embodiment 22: The method, oligonucleotide set, or combination of components of any one of the preceding embodiments, wherein at least one complementary pair of modified bases includes modified forms of adenine and thymine.

Embodiment 23: The method, oligonucleotide set, or combination of components of embodiment 22, wherein the modified forms of adenine and thymine are 2-aminoadenine and 2-thiothymine, respectively.

Embodiment 24: The method, oligonucleotide set, or combination of components of any one of the preceding embodiments, wherein at least one complementary pair of modified bases includes modified forms of guanine and cytosine.

Embodiment 25: The method, oligonucleotide set, or combination of components of embodiment 24, wherein the modified forms of guanine includes deoxyinosine, 7-alkyl-7-deazaguanine, 2′-hypoxanthine, or 7-nitro-7-deazahypoxanthine, and the modified form of cytosine includes 3-(2?-deoxy-beta-D-ribofuranosyl)pyrrolo-[2,3-d]-pyrimidine-2-(3H)-one, N4-alkylcytosine, or 2-thiocytosine.

Embodiment 26: The method, oligonucleotide set, or combination of components of any one of the preceding embodiments, wherein the blocker oligonucleotide and the capture oligonucleotide each comprise at least 2, 3, 4, 5, 6, 7, 8, 9, or 10 modified bases.

Embodiment 27: The method, oligonucleotide set, or combination of components of any one of the preceding embodiments, wherein the blocker oligonucleotide is blocked to 3′ extension.

### BRIEF DESCRIPTION OF THE DRAWINGS

FIG. 1A: A schematic drawing showing an amplification-based assay for the presence of one sequence (e.g., the mutant sequence) in the presence of a blocking oligonucleotide that destabilizes hybridization of the second sequence (e.g., the wild-type sequence). The blocker oligonucleotide preferentially blocks amplification of wild-type sequences to allow better detection of mutations.

FIG. 1B: A schematic drawing showing how excess blocker oligonucleotide carried over into hybridization-based mutation detection can interfere with capture of the amplified, mutant DNA. In this case, the capture oligonucleotide is the probe for the mutant sequence. Note: if the other strand were to be captured, the blocker oligonucleotide would interfere by binding to it.

FIG. 2: A schematic drawing showing how the use of modified (e.g., pseudo-complementary bases in the blocker and capture oligonucleotides reduce or prevent the interference shown in FIG. 1B. (Modified bases are identified with as A′ and T′ corresponding to modified (e.g., pseudo-complementary) forms of adenine and thymine.

FIG. 3: A schematic drawing showing common next-generation DNA sequencing protocols for DNA and RNA.

FIG. 4A: Base-pairing schemes for Watson-Crick doublets between thymine and adenine (Formula 1a), thymine and 2-aminoadenine (Formula 1b), 2-thiothymine and adenine (Formula 2b), and 2-thiothymine and 2-aminoadenine (Formula 2b). The 2-thiothymine and 2-aminoadenine base pair is destabilizing, whereas the thymine and 2-aminoadenine and the 2-thiothymine and adenine base pairs are stabilizing.

FIG. 4B: Base-pairing schemes for Watson-Crick doublets between cytosine and guanine (Formula 3a), cytosine and inosine (Formula 3b), dP and guanine (Formula 4a), and dP and inosine (Formula 4b). The dP and inosine base pair is destabilizing, whereas the cytosine and inosine and the dP and guanine base pairs are stable.

FIG. 5: Results from a liquid-phase hybridization assay demonstrating that, in testing desirable hybridization between a 3′ labeled fluorescent capture oligonucleotide and complementary oligonucleotides with either a 3′ or 5′ attached quencher, the 5′ quencher performed better. The results are from a Melt analysis run on QUANTSTUDIO 7. The analysis employed 300 nM fluorescent, biotinylated oligonucleotide (the biotin was not required in the present assay, but the available oligonucleotide happened to be biotinylated) with 3000 nM quencher oligonucleotide. The absence of salts in the assay led to a lower Tₘ, than would occur in PCR, (See Example 1,)

FIG. 6A: The same assay as shown in FIG. 5 was conducted with 300 nM 3′ labeled fluorescent capture oligonucleotide, 300 nM 5′ quencher oligonucleotide and 3000 nM, 300 nM, 30 nM, or 0 nM of pseudo-complementary blocker oligonucleotide. The results show that the pseudo-complementary blocker did not interfere with hybridization of the quencher oligonucleotide to a pseudo-complementary capture oligonucleotide.

FIG. 6B: Results from an assay identical in format to that of 6A, except that a blocker oligonucleotide that did not contain any modified bases (“complementary blocker”) interfered with hybridization of the quencher oligonucleotide to a pseudo-complementary capture oligonucleotide.

### DETAILED DESCRIPTION

### Definitions

Terms used in the claims and specification are defined as set forth below unless otherwise specified.

The term “nucleic acid” refers to a nucleotide polymer, and unless otherwise limited, includes analogs of natural nucleotides that can function in a similar manner (e.g., hybridize) to naturally occurring nucleotides.

The term nucleic acid includes any form of DNA or RNA, including, for example, genomic DNA; complementary DNA (cDNA), which is a DNA representation of mRNA, usually obtained by reverse transcription of messenger RNA (mRNA) or by amplification; DNA molecules produced synthetically or by amplification; mRNA; and non-coding RNA.

The term nucleic acid encompasses double- or triple-stranded nucleic acid complexes, as well as single-stranded molecules. In double- or triple-stranded nucleic acid complexes, the nucleic acid strands need not be coextensive (i.e., a double-stranded nucleic acid need not be double-stranded along the entire length of both strands).

The term nucleic acid also encompasses any modifications thereof, such as by methylation and/or by capping. Nucleic acid modifications can include addition of chemical groups that incorporate additional charge, polarizability, hydrogen bonding, electrostatic interaction, and functionality to the individual nucleic acid bases or to the nucleic acid as a whole. Such modifications may include base modifications such as 2′-position sugar modifications, 5-position pyrimidine modifications, 8-position purine modifications, modifications at cytosine exocyclic amines, substitutions of 5-bromo-uracil, sugar-phosphate backbone modifications, unusual base pairing combinations such as the isobases isocytidine and isoguanidine, and the like.

More particularly, in some embodiments, nucleic acids, can include polydeoxyribonucleotides (containing 2-deoxy-D-ribose), polyribonucleotides (containing D-ribose), and any other type of nucleic acid that is an N- or C-glycoside of a purine or pyrimidine base, as well as other polymers containing nonnucleotidic backbones, for example, polyamide (e.g., peptide nucleic acids (PNAs)) and polymorpholino polymers (see, e.g., Summerton and Weller (1997) “Morpholine Antisense Oligomers: Design, Preparation, and Properties,” Antisense & Nucleic Acid Drug Dev, 7:1817-195; Okamoto et al. (20020) “Development of electrochemically gene-analyzing method using DNA-modified electrodes,” Nucleic Acids Res. Supplement No. 2:171-172), and other synthetic sequence-specific nucleic acid polymers providing that the polymers contain nucleobases in a configuration which allows for base pairing and base stacking, such as is found in DNA and RNA. The term nucleic acid also encompasses locked nucleic acids (LNAs), which are described in U.S. Pat. Nos. 6,794,499, 6,670,461, 6,262,490, and 6,770,748, which are incorporated herein by reference in their entirety for their disclosure of LNAs.

The nucleic acid(s) can be derived from a completely chemical synthesis process, such as a solid phase-mediated chemical synthesis, from a biological source, such as through isolation from any species that produces nucleic acid, or from processes that involve the manipulation of nucleic acids by molecular biology tools, such as DNA replication, PCR amplification, reverse transcription, or from a combination of those processes.

As used herein, the term “complementary” refers to the capacity for precise pairing between two nucleotides; i.e., if a nucleotide at a given position of a nucleic acid is capable of hydrogen bonding with a nucleotide of another nucleic acid to form a canonical base pair, then the two nucleic acids are considered to be complementary to one another at that position. Complementarity between two single-stranded nucleic acid molecules may be “partial,” in which only some of the nucleotides bind, or it may be complete when total complementarity exists between the single-stranded molecules. The degree of complementarity between nucleic acid strands has significant effects on the efficiency and strength of hybridization between nucleic acid strands.

“Specific hybridization” refers to the binding of a nucleic acid to a target nucleotide sequence in the absence of substantial binding to other nucleotide sequences present in the hybridization mixture under defined stringency conditions. Those of skill in the art recognize that relaxing the stringency of the hybridization conditions allows sequence mismatches to be tolerated.

In some embodiments, hybridizations are carried out under stringent hybridization conditions. The phrase “stringent hybridization conditions” generally refers to a temperature in a range from about 5° C. to about 20° C. or 25° C. below than the melting temperature (Tₘ) for a specific sequence at a defined ionic strength and pH. As used herein, the Tₘ is the temperature at which a population of double-stranded nucleic acid molecules becomes half-dissociated into single strands. Methods for calculating the Tₘ of nucleic acids are well known in the art (see, e.g., Berger and Kimmel (1987) METHODS IN ENZYMOLOGY, VOL.152: GUIDE TO MOLECULAR CLONING TECHNIQUES, San Diego: Academic Press, Inc. and Sambrook et al. (1989) MOLECULAR CLONING: A LABORATORY MANUAL, 2ND ED., VOLS. 1-3, Cold Spring Harbor Laboratory), both incorporated herein by reference for their descriptions of stringent hybridization conditions). As indicated by standard references, a simple estimate of the Tₘ value may be calculated by the equation: Tₘ=81.5+0.41(% G+C), when a nucleic acid is in aqueous solution at 1 M NaCl (see, e.g., Anderson and Young, Quantitative Filter Hybridization in NUCLEIC ACID HYBRIDIZATION (1985)). The melting temperature of a hybrid (and thus the conditions for stringent hybridization) is affected by various factors such as the length and nature (I)NA, RNA, base composition) of the primer or probe and nature of the target nucleic acid (DNA, RNA, base composition, present in solution or immobilized, and the like), as well as the concentration of salts and other components (e.g., the presence or absence of formamide, dextran sulfate, polyethylene glycol). The effects of these factors are well known and are discussed in standard references in the art. Illustrative stringent conditions suitable for achieving specific hybridization of most sequences are: a temperature of at least about 60° C. and a salt concentration of about 0.2 molar at pH7. Tₘ calculation for oligonucleotide sequences based on nearest-neighbors thermodynamics can carried out as described in “A unified view of polymer, dumbbell, and oligonucleotide DNA nearest-neighbor thermodynamics” John SantaLucia, Jr., PNAS Feb. 17, 1998 vol. 95 no. 4 1460-1465 (which is incorporated by reference herein for this description).

The term “non-specific hybridization” is used herein to refer to hybridization between two nucleic acids (e.g., two oligonucleotides) that are less than fully complementary.

The term “oligonucleotide” is used to refer to a nucleic acid that is relatively short, generally shorter than 200 nucleotides, more particularly, shorter than 100 nucleotides, most particularly, shorter than 50 nucleotides. Typically, oligonucleotides are single-stranded DNA molecules.

The term “target nucleic acid” is used herein to refer to particular nucleic acid to be detected or sequenced in the methods described herein.

As used herein the term “target nucleic acid sequence” refers to a the nucleotide sequence of a target nucleic acid, such as, for example, the amplification product obtained by amplifying a target nucleic acid or the cDNA produced upon reverse transcription of an RNA target nucleic acid.

A “polymorphic marker” or “polymorphic site” is a locus at which nucleotide sequence divergence occurs. Illustrative markers have at least two alleles, each occurring at frequency of greater than 1%, and more typically greater than 10% or 20% of a selected population. A polymorphic site may be as small as one base pair. Polymorphic markers include restriction fragment length polymorphism (RFLPs), variable number of tandem repeats (VNTR's), hypervariable regions, minisatellites, dinucleotide repeats, trinucleotide repeats, tetranucleotide repeats, simple sequence repeats, deletions, and insertion elements such as Alu. The first identified allelic form is arbitrarily designated as the reference form and other allelic forms are designated as alternative or variant alleles. The allelic form occurring most frequently in a selected population is sometimes referred to as the “wild-type” form. Rarely occurring polymorphisms may be designated as “mutant” forms of a sequence. Mutant forms of a sequence can confer phenotypic difference on an organism, e.g., susceptibility to a disease or drug resistance in a pathogenic organism. Diploid organisms may be homozygous or heterozygous for allelic forms. A diallelic polymorphism has two forms. A triallelic polymorphism has three forms.

A “single nucleotide polymorphism” (SNP) occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations). A SNP usually arises due to substitution of one nucleotide for another at the polymorphic site. A transition is the replacement of one purine by another purine or one pyrimidine by another pyrimidine. A transversion is the replacement of a purine by a pyrimidine or vice versa. SNPs can also arise from a deletion of a nucleotide or an insertion of a nucleotide relative to a reference allele.

The term “primer” refers to an oligonucleotide that is capable of hybridizing (also termed “annealing”) with a nucleic acid and serving as an initiation site for nucleotide (RNA or DNA) polymerization under appropriate conditions (i.e., in the presence of four different nucleoside triphosphates and an agent for polymerization, such as DNA or RNA polymerase or reverse transcriptase) in an appropriate buffer and at a suitable temperature. The appropriate length of a primer depends on the intended use of the primer, but primers are typically at least 7 nucleotides long and, in some embodiments, range from 10 to 30 nucleotides, or, in some embodiments, from 10 to 60 nucleotides, in length. In some embodiments, primers can be, e.g., 15 to 50 nucleotides long. Short primer molecules generally require cooler temperatures to form sufficiently stable hybrid complexes with the template. A primer need not reflect the exact sequence of the template but must be sufficiently complementary to hybridize with a template.

A primer is said to anneal to another nucleic acid if the primer, or a portion thereof, hybridizes to a nucleotide sequence within the nucleic acid. The statement that a primer hybridizes to a particular nucleotide sequence is not intended to imply that the primer hybridizes either completely or exclusively to that nucleotide sequence. For example, in some embodiments, amplification primers used herein are said to “anneal to” or be “specific for” a nucleotide sequence.” This description encompasses primers that anneal wholly to the nucleotide sequence, as well as primers that anneal partially to the nucleotide sequence.

The term “primer pair” refers to a set of primers including a 5′ “upstream primer” or “forward primer” that hybridizes with the complement of the 5′ end of the DNA sequence to be amplified and a 3′ “downstream primer” or “reverse primer” that hybridizes with the 3′ end of the sequence to be amplified. As will be recognized by those of skill in the art, the terms “upstream” and “downstream” or “forward” and “reverse” are not intended to be limiting, but rather provide illustrative orientations in some embodiments.

A “probe” is a nucleic acid capable of binding to a target nucleic acid of complementary sequence through one or more types of chemical bonds, generally through complementary base pairing, usually through hydrogen bond formation, thus forming a duplex structure. The probe can be labeled with a detectable label to permit facile detection of the probe, particularly once the probe has hybridized to its complementary target. Alternatively, however, the probe may be unlabeled, hut may be detectable by specific binding with a ligand that is labeled, either directly or indirectly, Probes can vary significantly in size. Generally, probes are at least 7 to 15 nucleotides in length. Other probes are at least 20, 30, or 40 nucleotides long. Still other probes are somewhat longer, being at least 50, 60, 70, 80, or 90 nucleotides long. Yet other probes are longer still, and are at least 100, 150, 200 or more nucleotides long. Probes can also be of any length that is within any range bounded by any of the above values (e.g., 15-20 nucleotides in length).

The primer or probe can be perfectly complementary to the target nucleotide sequence or can be less than perfectly complementary. In some embodiments, the primer has at least 65% identity to the complement of the target nucleotide sequence over a sequence of at least 7 nucleotides, more typically over a sequence in the range of 10-30 nucleotides, and, in some embodiments, over a sequence of at least 14-25 nucleotides, and, in some embodiments, has at least 75% identity, at least 85% identity, at least 90% identity, or at least 95%, 96%, 97%, 98%, or 99% identity. It will be understood that certain bases (e.g., the 3′ base of a primer) are generally desirably perfectly complementary to corresponding bases of the target nucleotide sequence. Primer and probes typically anneal to the target sequence under stringent hybridization conditions.

As used herein with reference to a portion of a primer or a nucleotide sequence within the primer, the term “specific for” a nucleic acid, refers to a primer or nucleotide sequence that can specifically anneal to the target nucleic acid under suitable annealing conditions.

The term “adaptor” is used herein to refer to a nucleic acid that, in use, becomes appended to one or both ends of a nucleic acid, e.g., a nucleic acid fragment. An adaptor may be single-stranded, double-stranded, or may include single- and double-stranded portions. Illustrative adaptors include DNA sequencing adaptors that are added to nucleic acid fragments to facilitate DNA sequencing. Different DNA sequencing platforms typically require different adaptors.

The term “template,” as used with reference to DNA sequencing refers to a sequence that contains the necessary components to be sequenced. Thus, a template for DNA sequencing can include adaptors that provide nucleotide sequences that facilitate DNA sequencing, such as a DNA sequencing primer binding site and a barcode nucleotide sequence.

The term “DNA sequencing primer binding site” is used herein to refer to a site to which a DNA sequencing primer anneals in a DNA sequencing template. At least one DNA sequencing primer binding site is oriented a template such that it primes synthesis of the portion of the template whose sequence is to be determined.

The term “barcode nucleotide sequence” is a sequence that encodes an item of information about a larger nucleotide sequence in which it appears. For example, a barcode nucleotide sequence could encode information about the sample or individual cell that a target nucleotide sequence was obtained from. Alternatively, a barcode nucleotide sequence can be a unique molecular identifier, meaning that each different target nucleotide sequence in a set of target nucleotide sequences has its own unique barcode nucleotide sequence.

Amplification according to the present teachings encompasses any means by which at least a part of at least one target nucleic acid is reproduced, typically in a template-dependent manner, including without limitation, a broad range of techniques for amplifying nucleic acid sequences, either linearly or exponentially. Illustrative means for performing an amplifying step include PCR, nucleic acid strand-based amplification (NASBA), two-step multiplexed amplifications, rolling circle amplification (RCA), and the like, including multiplex versions and combinations thereof, for example but not limited to, OLA/PCR, PCR/OLA, LDR/PCR, PCR/PCR/LDR, PCR/LDR, LCR/PCR, PCR/LCR (also known as combined chain reaction-CCR), helicase-dependent amplification (HDA), and the like. Descriptions of such techniques can be found in, among other sources. Ausubel et al.; PCR Primer: A Laboratory Manual, Diffenbach, Ed., Cold Spring Harbor Press (1995); The Electronic Protocol Book, Chang Bioscience (2002); Msuih et al., J. Clin. Micro. 34:501-07 (1996); The Nucleic Acid Protocols Handbook, R. Rapley, ed., Humana Press, Totowa, N.J. (2002); Abramson et al., Curr Opin Biotechnol. 1993 February; 4(1):41-7, U.S. Pat. Nos. 6,027,998; 6,605,451, Barany et al., Publication No. WO 97/31256; Wenz et al., PCT Publication No. WO 01/112579; Day et al., Genomics, 29(1): 152-162 (1995), Ehrlich et al., Science 252:1643-50 (1991); Innis et al., PCR Protocols: A Guide to Methods and Applications, Academic Press (1990); Favis et al., Nature Biotechnology 18:561-64 (2000); and Rabenau et al., Infection 28:97-102 (2000); Belgrader, Barany, and Lubin., Development of a Multiplex Ligation Detection Reaction DNA Typing Assay, Sixth International Symposium on Human Identification, 1995 (available on the world wide web at: promega.com/geneticidproc/ussymp6proc/blegrad.html-); LCR Kit Instruction Manual, Cat. #200520, Rev. #050002, Stratagene, 2002; Barany, Proc. Natl. Acad. Sci. USA 88:188-93 (1991); Bi and Sambrook, Nucl. Acids Res. 25:2924-2951 (1997); Zirvi et al., Nucl. Acid Res. 27:e40i-viii (1999); Dean et al., Proc Natl. Acad Sci USA 99:5261-66 (2002); Barany and Gelfand, Gene 109:1-11 (1991); Walker et al., Nucl. Acid Res. 20:1691-96 (1992); Polstra et al., BMC Inf. Dis. 2:18-(2002); Lage et al., Genome Res. 2003 February; 13(2):294-307, and Landegren et al., Science 241:1077-80 (1988), Demidov, V., Expert Rev Mol Diagn. 2002 November; 2(6):542-8., Cook et al., J Microbiol Methods. 2003 May; 53(2):165-74, Schweitzer et al., Curr Opin Biotechnol. 2001 February; 12(1): 1-7, U.S. Pat. Nos. 5,830,711, 6,027,889, 5,686,243, PCT Publication No. WO0056927A3, and PCT Publication No. WO9803673A1.

In some embodiments, amplification comprises at least one cycle of the sequential procedures of: annealing at least one primer with complementary or substantially complementary sequences in at least one target nucleic acid; synthesizing at least one strand of nucleotides in a template-dependent manner using a polymerase; and denaturing the newly-formed nucleic acid duplex to separate the strands. The cycle may or may not be repeated. Amplification can comprise thermocycling or can be performed isothermally.

As used herein, the term “support” refers to any substrate, typically one to which oligonucleotides can be attached. If oligonucleotides are attached to the support, the support is generally non-reactive to other components that will contact the support in use. The support can be insoluble (e.g., a planar surface or a microbead) or soluble (e.g., a water-soluble polymer that can easily be removed from a reaction mixture by, e.g., centrifugation and/or precipitation).

As used herein, the term “microbead” refers to a bead having a diameter that is less than 1 mM (i.e., less than 1000 microns). Microbeads may be microscopic or near-microscopic and may have diameters of about 0.005 to 100 μm, about 0.1 to 50 μm, or about 0.5 to 30 μm.

A “multiplex amplification reaction” is one in which two or more nucleic acids distinguishable by sequence are amplified simultaneously.

The term “qPCR” is used herein to refer to quantitative real-time polymerase chain reaction (PCR), which is also known as “real-time PCR” or “kinetic polymerase chain reaction;” all terms refer to PCR with real-time signal detection.

A “reagent” refers broadly to any agent used in a reaction, other than the analyte (e.g., nucleic acid being analyzed). Illustrative reagents for a nucleic acid amplification reaction include, but are not limited to, buffer, metal ions, polymerase, reverse transcriptase, primers, template nucleic acid, nucleotides, labels, dyes, nucleases, dNTPs, and the like. Reagents for enzyme reactions include, for example, substrates, cofactors, buffer, metal ions, inhibitors, and activators.

The term “label,” as used herein, refers to any atom or molecule that can be used to provide a detectable and/or quantifiable signal. In particular, the label can be attached, directly or indirectly, to a nucleic acid or protein. Suitable labels that can be attached to probes include, but are not limited to, radioisotopes, fluorophores, chromophores, mass labels, electron dense particles, magnetic particles, spin labels, molecules that emit chemiluminescence, electrochemically active molecules, enzymes, cofactors, and enzyme substrates.

The term “dye,” as used herein, generally refers to any organic or inorganic molecule that absorbs electromagnetic radiation.

The naturally occurring bases adenine, thymine, uracil, guanine, and cytosine, which make up DNA and RNA, are described herein as “unmodified bases” or “unmodified forms.”

The term “modified base” is used herein to refer to a base that is not a canonical, naturally occurring base (e.g., adenine, cytosine, guanine, thymine, or uracil). Examples of modified bases are the pseudo-complementary bases 2-thiothymine and 2-aminoadenine.

Nucleotides including modified bases are referred to herein as “modified nucleotides” (e.g., pseudo-complementary nucleotides).

Oligonucleotides including one or more “modified nucleotides” (e.g., pseudo-complementary nucleotides) are referred to herein as pseudo-complementary oligonucleotides (e.g., pseudo-complementary blocker oligonucleotide).

### Methods of Determining Whether a Nucleotide Sequence is Present

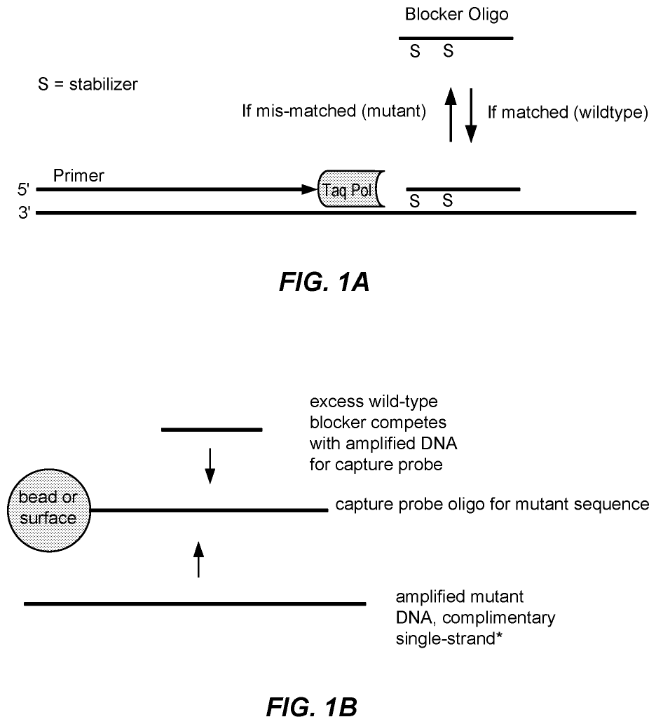

The present disclosure provides a method of determining whether a particular a nucleotide sequence is present in a target nucleic acid sequence in a sample, where the target nucleic acid sequence comprises a polymorphic site, such as a single nucleotide polymorphism. One way of enhancing the discrimination between two possible sequences, e.g., a wild-type sequence and a mutant sequence, is to assay for the presence of one sequence (e.g., the mutant sequence) in the presence of a blocking oligonucleotide that destabilizes hybridization of the second sequence (e.g., the wild-type sequence). Such an assay is shown in FIG. 1A.

This approach has been used, for example, in a method termed “wild-type blocking-polymerase chain reaction” (WTB-PCR), which is described, for example, Dominguez (2005) Oncogene 24:6830-6834 and in U.S. Pat. No. 10,227,657 (issued Mar. 19, 2019 to Maher et al.). Briefly, WTB-PCR was developed to facilitate the detection and sequencing of minority mutations from clinical specimens. In WTB-PCR, a non-extendable locked nucleic acid (LNA) oligonucleotide binds tightly to a region of wild-type DNA known to develop point mutations. This blocker oligonucleotide inhibits primer extension through the polymorphic region for the wild-type allele, whereas primer extension through the polymorphic region proceeds normally for the mutant allele to produce amplified mutant DNA (see FIG. 1A), This technique allows sensitive detection of minority mutations in a tissue sample containing excess wild-type DNA.

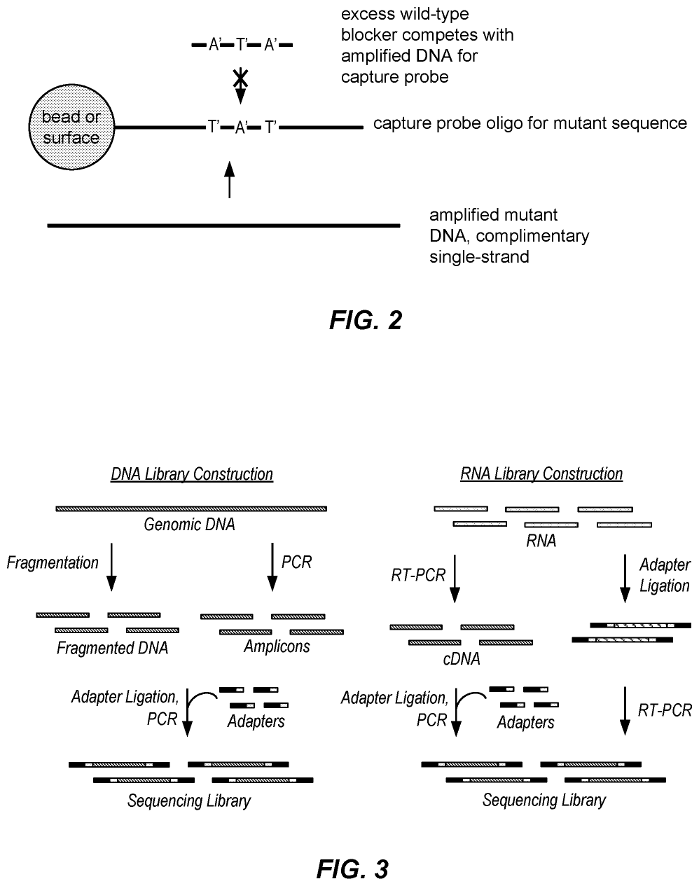

In some embodiments, the amplification product from WTB-PCR is hybridized to a capture oligonucleotide for detection. In such cases, blocker oligonucleotide remaining in the amplification product mixture can compete with the amplified mutant DNA for hybridization to the capture oligonucleotide, as shown in FIG. 2B. The competition can prevent the detection of a mutant allele that is present in a sample.

The present method overcomes this difficulty by using a blocker oligonucleotide and capture oligonucleotide pair that each include one or more pseudo-complementary bases. The one or more pseudo-complementary bases are positioned so that, if the blocker oligonucleotide were to hybridize to the capture oligonucleotide, at least one (and preferably more) pseudo-complementary base(s) in the blocker oligonucleotide would be faced with pairing with its complementary pseudo-complementary base. Since this pairing is disfavored, relative to normal Watson-Crick base pairing, the degree to which the blocker oligonucleotide can compete with the amplified mutant DNA for binding to the capture oligonucleotide is reduced, improving the sensitivity of the assay for the mutant allele.

The blocker oligonucleotide can, but need not, include LNAs. In some embodiments, is blocked to 3′ extension, e.g., by virtue of lacking a 3′ hydroxyl group or using a chemical blocking moiety. This modification can improve the specificity of the amplification.

The present method is described in terms of a “wild-type” allele and a “mutant” allele for ease of understanding, but those of skill in the are readily appreciate that this method is applicable to the detection of one of at least two possible forms of a sequence, such as, e.g., the detection of one of two alleles that do not have a wild-type-mutant relationship.

The pseudo-complementary bases typically replace residues at sequence positions that are common between two forms of the sequence. The number of pseudo-complementary bases used in the blocker and capture oligonucleotides will usually be the same (although this is not a requirement of the method). The number used can vary, depending upon the length of the complementary sequences in the oligonucleotides. In general, longer oligonucleotides can require the use of more pseudo-complementary bases than shorter oligonucleotides because it may take more pseudo-complementary bases to adequately destabilize blocker and capture oligonucleotide hybridization. The degree of destabilization required is the degree that sufficiently reduces competition of the blocker oligonucleotide with amplified DNA for binding to the capture oligonucleotide. These parameters can be determined empirically based on the guidance provided herein. In particular, Example 1 shows how oligonucleotides can be tested for a reduction in binding competition by the blocker oligonucleotide. In this Example, three pseudo-complementary bases were used in a 16-nucleotide blocker oligonucleotide. In illustrative embodiments, at least 5%, 10%, 15%, 16%, 17%, 18, %, 19%, 20%, 21%, 22%, 23%, 24%, 25%, 30%, 35%, 40%, 45%, 50%, 55%, 60%, 65%, 70%, 75%, 80%, 85%, 90%, 95%, or all of the bases in a blocker and/or capture oligonucleotide can be pseudo-complementary bases. In various embodiments, the number of pseudo-complementary bases in a blocker and/or capture oligonucleotide falls within a range bounded by any of these values, e.g., 10%-40%, 15%-35%, 16%-30%, 17%-25%, or 18%-20%.

In certain embodiments, the capture oligonucleotide can be attached to a support. In illustrative embodiments, the support can be an insoluble support, such as a planar surface or a microbead or a soluble support, such as a water-soluble polymer than can easily be recovered from a reaction mixture by, e.g. centrifugation and/or precipitation. In general, assay format will dictate whether, and what type of support should be used.

In some embodiments, the method of determining whether a nucleotide sequence is present also includes quantifying the amount (relative or absolute) of the nucleotide sequence. In certain embodiments, a probe can be used for detection/quantification.

The method can be used to assay a sample comprising a small minority (less than 50%) of cells of one type (e.g., cancerous cells) in a large background of cells of a different type. In various embodiments, the minority of cells is less than approximately 45%, 40%, 35%, 30%, 25%, 20%, 15%, 10%, 5%, 4%, 3%, 2%, 1%, 0.5%, 0.1%, 0.05%, 0.01%, 0.005%, 0.001%, 0.0005%, or 0.0001% of the total number of cells. In various embodiments, the percentage of minority cells falls within a range bounded by any of the values, e.g., 10%-0.0001%, 5%-0.001%, 1% to 0.01%, or 0.5% to 0.1%.

In some embodiments the methods described herein can be used to assay single cells.

### Nucleic Acid Sequencing Methods

The present disclosure provides a method for simplifying preparations for nucleic acid sequencing that find particular application in next-generation sequencing protocols. For reviews of next-generation sequencing and sequencing library generation, see Goodwin et al. (2016) “Coming of Age: Ten Years of next-Generation Sequencing Technologies.” Nature Reviews Genetics 17(6): 333-51 and Head et al. (2014) “Library Construction for next-Generation Sequencing: Overviews and Challenges.” BioTechniques 56(2): 61.

Briefly, “sequencing by synthesis” is perhaps the most well-established next-generation sequencing method, and is used by the 454, Illumina, Qiagen, and Ion Torrent (Thermo Fisher) platforms, with each platform utilizing their own technologies, Instrument models within a platform may come in varying levels of sequencing capabilities and throughput. Sample loading chips and kits for a given instrument may also be scalable to feature additional higher-throughput options. Those of skill in the art are familiar with these platforms and with which to select for different applications. For example, whole-genome or whole-transcriptome sequencing may require higher throughputs, and de novo sequencing and metagenomic sequencing may benefit from longer read lengths.

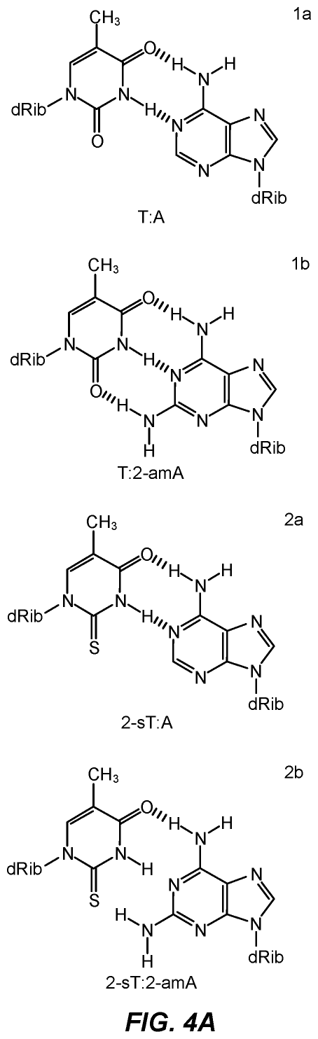

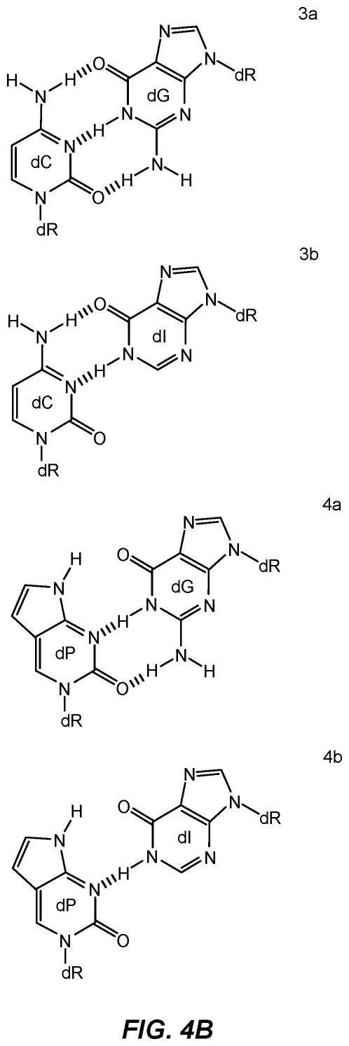

The typical sample preparation workflow for next-generation sequencing (NGS), shown schematically in FIG. 3, can include: (1) nucleic acid fragmentation or amplification to produce nucleic acid fragments suitably sized for sequencing (e.g., typically used for DNA); (2) cDNA synthesis for RNA, (3) addition of sequencing adaptors (to DNA or RNA), typically by ligation (DNA or RNA ligases can be used to add adaptors to DNA or RNA, respectively; (4) amplification (e.g., PCR), (5) target enrichments, and (6) quantification.

The sequencing library fragment size depends mainly on the desired insert size (between the adaptors) and the limitations of the NGS platform. Illumina's cluster amplification step following adapter ligation can accommodate a range of up to 1500 bp. For Ion Torrent, fragment sizes of 100 to 600 bp should be suitable. Commercial kits are available for enzymatic fragmentation that specify one sequencing platform and detail fragmentation size outputs.

Kits are available for RNA sequencing applications that include reagents for reverse transcription into cDNA, either by PCR or PCR-free. Some also feature enrichment for specific RNA types, either by capturing mRNA or depleting rRNA. These allow for streamlined library construction directly from RNA samples ranging from inputs of 25 to 1000 ng.

Commercial enzyme kits for adapter ligation contain reagents tailored to the sequencing platform. The general workflow involves end repair of the DNA fragments followed by ligation of platform-specific adaptors. The major difference between Illumina and Ion Torrent is that the latter uses blunt-end ligation. Kits typically include all the enzymes (such as ligases and polymerases) and buffers necessary, and some feature additional barcodes for multiplexing.

For either Illumina or Ion Torrent platforms, many commercial library preparation kits include PCR polymerases for subsequent amplification following adapter ligation. Some feature high-fidelity and hot-start polymerases for improved coverage and lower duplication rates.

The large amount of data generated by whole genome sequencing, for example, can complicate data processing and analysis. As a workaround, portions of genomes may be enriched to focus on key genes. With target enrichment, DNA segments can be enriched either by hybridization-based capture or multiplex PCR. Several kits are available for the in-solution capture using biotinylated RNA or oligonucleotide probes that bind to streptavidin beads.

Prior to the sequencing run, sequencing libraries may require accurate quantification to ensure good data output and quality. NGS quantification kits are available that utilize qPCR, which are selective for the molecules with the right adaptor sequences. For convenience and consistency, kits include complete sets of reagents and some feature prediluted DNA standards.

The present method simplifies library preparation for those workflows in which amplified DNA templates are hybridized to oligonucleotides, which are typically attached to a solid support, Examples include oligonucleotides to which “paired-end” sequences flanking a DNA template hybridize Illumina's flow cells. These oligonucleotides are termed “capture oligonucleotides” for the purposes of the present discussion. The design considerations for capture oligonucleotides used in a sequencing method are similar to those described above for capture oligonucleotides used in determining whether a particular nucleotide sequence is present, except that capture oligonucleotides include pseudo-complementary bases to reduce the likelihood that free adaptor sequences will hybridize to the capture oligonucleotides, instead of the desired hybridization between the capture oligonucleotides and the amplified DNA templates. This problem arises because, typically, the sequences that allow the amplified DNA templates to hybridize to the capture oligonucleotides are present in the adaptor sequences.

In the present method, competition between adaptors and amplified DNA templates for binding capture oligonucleotides is reduced or eliminated by including pseudo-complementary bases in the adaptors and in corresponding positions in the capture oligonucleotides. The considerations for reducing or eliminating adaptor binding to capture oligonucleotides are essentially the same as those discussed above for reducing blocker oligonucleotide to capture oligonucleotides (see also below, the section entitled “Primer/Probe/Blocker Oligonucleotide/Adaptor./-Capture Oligonucleotide Design”). The method works because free adaptors contain pseudo-complementary bases which will not pair efficiently with their counterparts in the capture oligonucleotide(s), whereas amplified DNA templates include the natural bases provided to the amplification reaction, which will pair relatively normally with the pseudo-complementary bases in the capture oligonucleotides.

### Samples

Nucleic acid-containing samples can be obtained from biological sources and prepared using conventional methods known in the art. In particular, nucleic useful in the methods described herein can be obtained from any source, including unicellular organisms and higher organisms such as plants or non-human animals, e.g., canines, felines, equines, primates, and other non-human mammals, as well as humans. In some embodiments, samples may be obtained from an individual suspected of being, or known to be, infected with a pathogen, an individual suspected of having, or known to have, a disease, such as cancer, or a pregnant individual.

Nucleic acids can be obtained from cells, bodily fluids (e.g., blood, a blood fraction, urine, etc.), or tissue samples by any of a variety of standard techniques. In some embodiments, the method employs samples of plasma, serum, spinal fluid, lymph fluid, peritoneal fluid, pleural fluid, oral fluid, and external sections of the skin; samples from the respiratory, intestinal genital, or urinary tracts; samples of tears, saliva, blood cells, stem cells, or tumors, Samples can be obtained from live or dead organisms or from in vitro cultures. Illustrative samples can include single cells, paraffin-embedded tissue samples, and needle biopsies. In some embodiments, the nucleic acids analyzed are obtained from a single cell.

Nucleic acids of interest can be isolated using methods well known in the art. The sample nucleic acids need not be in pure form, but are typically sufficiently pure to allow the steps of the methods described herein to be performed.

### Target Nucleic Acids

Any target nucleic acid that can detected by nucleic acid amplification can be detected or sequenced using the methods described herein. In some embodiments, at least some nucleotide sequence information will be known for the target nucleic acids. For example, if the amplification reaction employed is PCR, sufficient sequence information is generally available for each end of a given target nucleic acid to permit design of suitable amplification primers. In nucleic acid sequencing embodiments, there may be no sequence information known for the “target nucleic acids,” which in this case are sequencing templates, if the sequencing templates are produced by adding DNA sequencing adaptors to both ends of nucleic acid fragments, since primers that bind in the adaptor sequences can be used for amplification.

The targets can include, for example, nucleic acids associated with pathogens, such as viruses, bacteria, protozoa, or fungi; RNAs, e.g., those for which over- or under-expression is indicative of disease, those that are expressed in a tissue-or developmental-specific manner; or those that are induced by particular stimuli; genomic DNA, which can be analyzed for specific polymorphisms (such as SNPs), alleles, or haplotypes, e.g., in genotyping. Of particular interest are genomic DNAs that are altered (e.g., amplified, deleted, and/or mutated) in genetic diseases or other pathologies; sequences that are associated with desirable or undesirable traits; and/or sequences that uniquely identify an individual (e.g., in forensic or paternity determinations).

### Primer/Probe/Blocker Oligonucleotide/Adaptor/Capture Oligonucleotide Design

Those of skill in the art are well-versed in the considerations associated with designing an oligonucleotide that is intended to anneal or hybridize (or not) to another nucleotide sequence in an assay. These considerations are discussed briefly below in terms of primers, and most of these considerations apply to other annealing/hybridizing oligonucleotides.

Primers suitable for nucleic acid amplification are sufficiently long to prime the synthesis of extension products in the presence of a suitable nucleic acid polymerase. The exact length and composition of the primer will depend on many factors, including, for example, temperature of the annealing reaction, source and composition of the primer, and where a probe is employed, proximity of the probe annealing site to the primer annealing site and ratio of primer:probe concentration. For example, depending on the complexity of the target nucleic acid sequence, an oligonucleotide primer typically contains in the range of about 10 to about 60 nucleotides, although it may contain more or fewer nucleotides. The primers should be sufficiently complementary to selectively anneal to their respective strands and form stable duplexes.

In general, one skilled in the art knows how to design suitable primers capable of amplifying a target nucleic acid of interest. For example, PCR primers can be designed by using any commercially available software or open source software, such as Primer3 (see, e.g., Dozen and Skaletsky (2000) Meth. Mol. Biol., 132: 365-386; www.broad.mitedu/node/1060, and the like) or by accessing the Roche UPL website. The amplicon sequences are input into the Primer3 program with the UPI, probe sequences in brackets to ensure that the Primer3 program will design primers on either side of the bracketed probe sequence.

The Tₘ of hybrids formed by primers, or any other oligonucleotides in an assay can be adjusted by including stabilizing or destabilizing bases in the primer/oligonucleotide.

“Stabilizing bases” include, e.g., stretches of peptide nucleic acids (PNAs) that can be incorporated into DNA oligonucleotides to increase duplex stability. Locked nucleic acids (LNAs) and unlocked nucleic acids (UNAs) are analogues of RNA that can be easily incorporated into DNA oligonucleotides during solid-phase oligonucleotide synthesis, and respectively increase and decrease duplex stability. Suitable stabilizing bases also include modified DNA bases that increase the stability of base pairs (and therefore the duplex as a Whole). These modified bases can be incorporated into oligonucleotides during solid-phase synthesis and offer a more predictable method of increasing DNA duplex stability. Examples include AP-dC (G-clamp) and 2-aminoadenine, as well as 5-methylcytosine and C(5)-propynylcytosine (replacing cytosine), and C(5)-propynyluracil (replacing thymine).

“Destabilizing bases” are those that destabilize double-stranded DNA by virtue of forming less stable base pairs than the typical A-T and/or G-C base pairs. Inosine (I) is a destabilizing base because it pairs with cytosine (C), but an I-C base pair is less stable than a G-C base pair. This lower stability results from the fact that inosine is a purine that can make only two hydrogen bonds, compared to the three hydrogen bonds of a G-C base pair. Other destabilizing bases are known to, or readily identified by, those of skill in the art.

As discussed above, the present methods are concerned with reducing unwanted hybridization between oligonucleotides (e.g., a blocker oligonucleotide and a capture oligonucleotide). Unwanted hybridization between any two oligonucleotides in an assay can be reduced or prevented by including pseudo-complementary bases in the oligonucleotides. Pseudo-complementary bases are described as “modified bases” in the next section.

Primers and other oligonucleotides may be prepared by any suitable method, including, for example, direct chemical synthesis by methods such as the phosphotriester method of Narang et al. (1979) Meth. Enzymol. 68: 90-99; the phosphodiester method of Brown et al. (1979) Meth. Enzymol. 68: 109-151; the diethylphosphoramidite method of Beaucage et al. (1981) Tetra. Lett., 22: 18594862; the solid support method of U.S. Pat. No. 4,458,066 and the like, or can be provided from a commercial source. Primers may be purified by using a Sephadex column (Amersham Biosciences, Inc., Piscataway, N.J.) or other methods known to those skilled in the art. Primer purification may improve the sensitivity of the methods described herein.

### Modified Bases

Modified bases useful in the primers and other oligonucleotides described herein include those wherein the modified base forms stable hydrogen-bonded base pairs with the natural complementary base but does not form stable hydrogen-bonded base pairs with its modified complementary base (e.g., pseudo-complementary bases). (For ease of discussion, complementary bases are also referred to herein as “partners.” Also, for ease of discussion, the following description relates to primers and primer pairs, but, as those of skill in the art, readily appreciate, this description also applies to the other oligonucleotides and oligonucleotide pairs described herein.) In some embodiments, this is accomplished when the modified base can form two or more hydrogen bonds with its natural partner, but only one or no hydrogen bonds with its modified partner. This allows the production of primer and other oligonucleotide pairs that do not form substantially stable hydrogen-bonded hybrids with one another, as manifested in a melting temperature (under physiological or substantially physiological conditions) of less than about 40° C. The primers of the primer pair, however, form substantially stable hybrids with the complementary nucleotide sequence in a template strand (e.g., first template strand) of a single- or double-stranded target nucleic acid and with a strand complementary to the template strand (e.g., second template strand). In some embodiments, due to the increased (in some embodiments, double) number of hydrogen bonds in such hybrids, the hybrids formed with the primers of the present invention are more stable than hybrids that would be formed using primers with unmodified bases.

In accordance with well-established convention, the naturally occurring nucleotides of nucleic acids have the designation A, U, G and C, (RNA) and dA, dT, dG and dC (DNA). The following description applies to both ribonucleotides and deoxyribonucleotides, and therefore, unless the context otherwise requires, no distinction needs to be made in this description between A and dA, U and dT, etc.

Analogs of A that are modified in the base portion to form a stable hydrogen-bonded pair with T, (or U in the case of RNA) but not with a modified T are designated A*. Analogs of T that are modified in the base portion to form a stable hydrogen-bonded pair with A, hut not with A* are designated T*. Analogs of G that are modified in the base portion to form a stable hydrogen-bonded pair with C, but not with a modified C are designated G*. Analogs of C that are modified in the base portion to form a stable hydrogen-bonded pair with G, but not with G* are designated C*, In some embodiments, the foregoing conditions are satisfied when each of the A*, T*, G*, and C* nucleotides (collectively, the modified nucleotides) form two or more hydrogen bonds with their natural partner, but only one or no hydrogen bonds with their modified partner. This is illustrated by Formulas 1a, 1b, 2a, 2b, 3a, 3b, 4a and 4b below (and in FIG. 8A-8B), where the hydrogen bonding between natural A-T (or A-U in case of RNA) and G-C pairs, and hydrogen bonding between exemplary A*-T, T*-A, G*-C, C*-G, A*-T* and G*-C* pairs are illustrated.

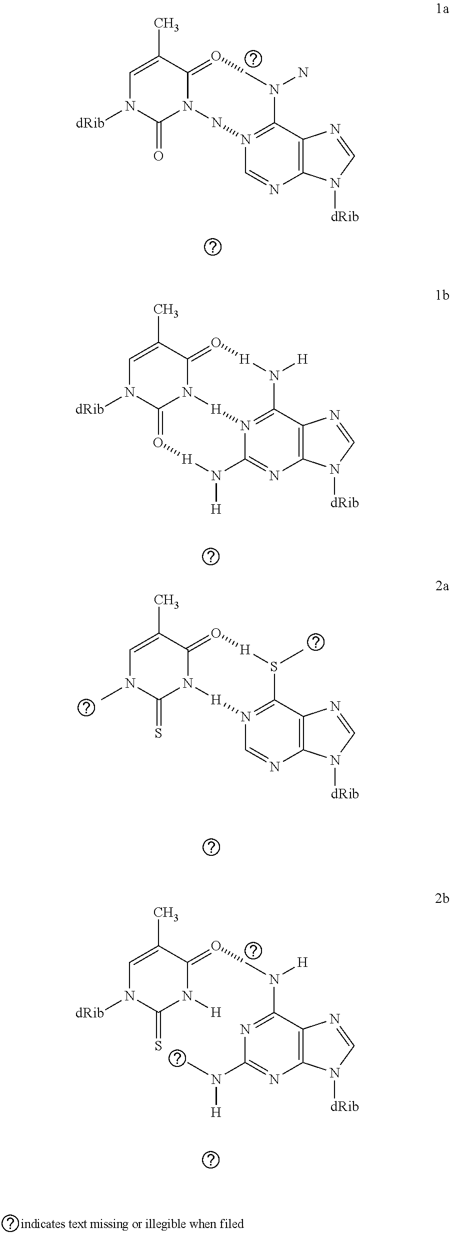

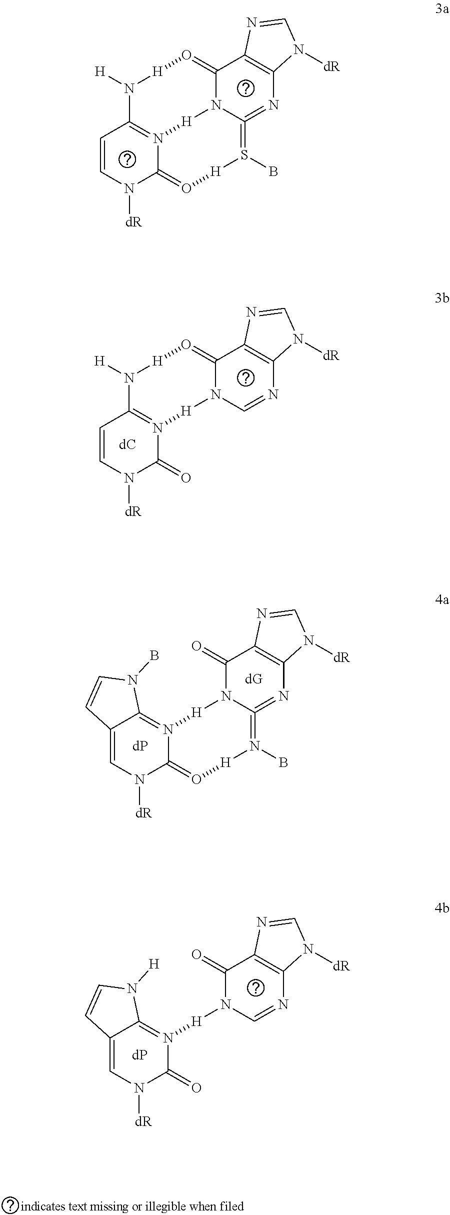

In general, a sufficient number of modified nucleotides are incorporated into the primers described herein to preferentially increase the annealing of the primers to the template strands of a target nucleic acid, as compared to primer-to-primer annealing. It is not necessary to replace each natural nucleotide of the primer with a modified nucleotide in order to accomplish this. In some embodiments, the primers include, in addition to one or more modified nucleotides, one or more naturally occurring nucleotides and/or variants of naturally occurring nucleotides, provided that the variations do not interfere significantly with the complementary binding ability of the primers, as discussed above. For example, primers including modified nucleotides can include pentofuranose moieties other than ribose or 2-deoxyribose, as well as derivatives of ribose and 2-deoxyribose, for example 3-amino-2-deoxyribose, 2-fluoro-2-deoxyribose, and 2—O—C₁₋₆ alkyl or 2-O-allyl ribose, particularly 2-O-methyl ribose. The glycosidic linkage can be in the α or β configuration. The phosphate backbone of the primer can, if desired, include phosphorothioate linkages.

A general structure for a suitable class of the modified A analog, A*, shown as a 3′-phosphate (or phosphorothioate) incorporated into a primer, is provided by Formulas 5, 6, and 7, below, wherein:

X is N or CH;

Y is O or S;

Z is OH or CH₃;

R is H, F, or OR₂, where R₂ is C₁₋₆alkyl or allyl, or H in case of RNA; and

R₁ is C₁₋₄alkyl, C₁₋₄ alkoxy, alkylthio, F, or NHR₃, where R₃ is H, or C₁₋₄ alkyl. An illustrative embodiment of A* has 2,6-diaminopurine (2-aminoadenine) as the base, as shown in Formula 1b. The latter nucleotide can be abbreviated as 2-amA or d2-amA, as applicable.

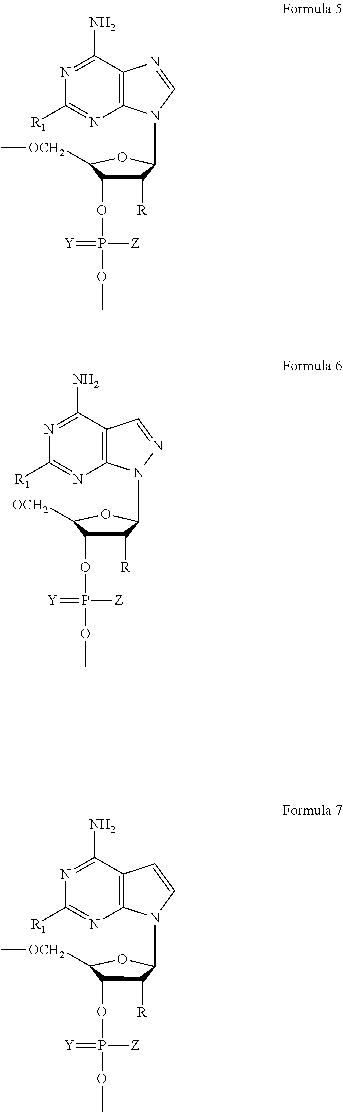

A general structure for a suitable class of the modified T analog, T*, shown as a 3′-phosphate (or phosphorothioate) incorporated into the primer, is provided by Formula 8, wherein:

Y, Z, and R are defined as above; and

R₄ is H, C₁₋₆ alkyl, C₁₋₆ alkenyl, or C₁₋₆ alkynyl. An illustrative embodiment of T* has 2-thio-4-oxo-5-methylpyrimidine (2-thiothymine) as the base, as shown in Formula 2b. The latter nucleotide can be abbreviated as 2-sT or d2-sT, as applicable.

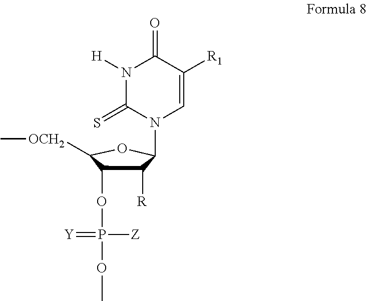

A general structure for a suitable class of the modified G analog, G*, shown as a 3°-phosphate (or phosphorothioate) incorporated into the primer, is provided by Formulas 9, 10 and 11, wherein:

R₁ is H, C₁₋₄ alkyl, C₁₋₄ alkoxy, C₁₋₄ alkylthio, F, or NHR₃, where R₃ is defined as above; and

X, Y, Z, and R are defined as above. An illustrative embodiment of G* has 6-oxo-purine (hypoxanthine) as the base, as shown in Formula 3b. The latter nucleotide can be abbreviated as I or dI, as applicable.

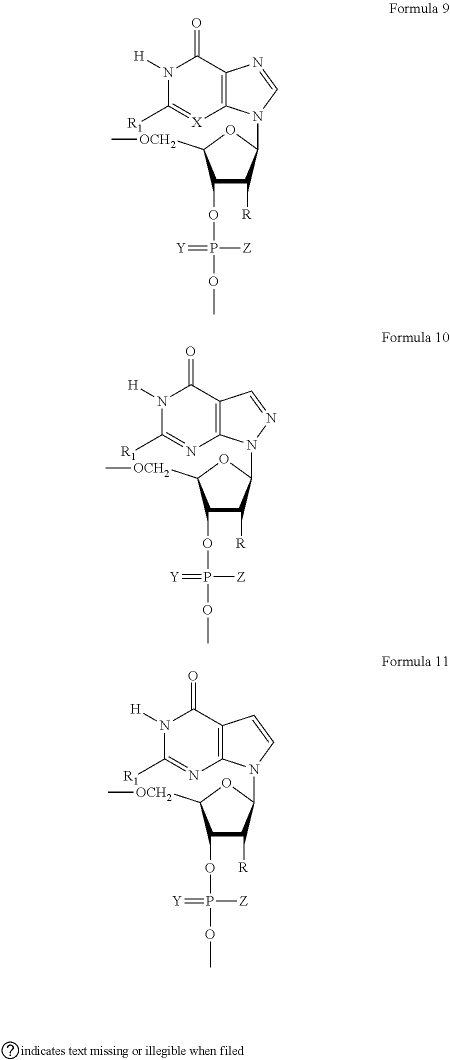

A general structure for a suitable class of the modified C analog, C*, shown as a 3′-phosphate (or phosphorothioate) incorporated into the primer, is provided by Formulas 12 and 13, wherein:

Y, Z, R, and R₄ are defined as above;

Z₁ is O or NH; and

R₅ is H or C₁₋₄ alkyl. An illustrative embodiment of C* has pyrrolo-[2,3-d]pyrimidine-2(3H)-one as the base, as shown in Formula 4b. The latter nucleotide can be abbreviated as P or dP, as applicable.

The above-described modified bases and nucleotides are also described in U.S. Pat. No. 5,912,340 (issued Jun. 15, 1999 to Kutyavin et al.), which is hereby incorporated by reference for this description. The hybridization properties of d2-amA and d2-sT are described in Kutyavin, et al. (1996) Biochemistry 35:11170-76, which is also hereby incorporated by reference for this description. The synthesis and hybridization properties of d/I and dP are described in Woo et al. (1996) Nucleic Acids Research 25(13):2470-75, which is also hereby incorporated by reference for this description.

Additional examples of G* and C* include 7-alkyl-7-deazaguanine and N⁴-alkylcytosine (where alkyl methyl or ethyl), respectively, which are described in Lahoud et al. (2008) Nucleic Acids Research 36(10):3409-19 (hereby incorporated by reference for this description). Analogs tested in this study are shown in Formula 12,

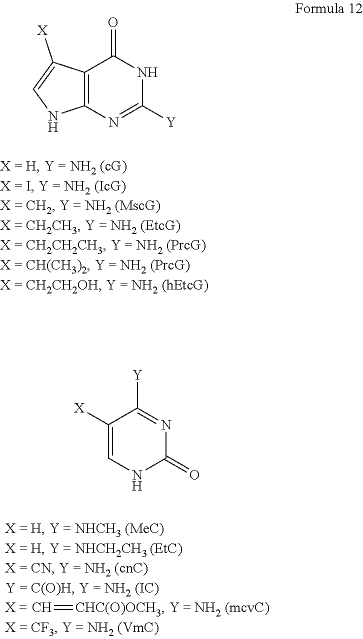

Further examples of G* and C* include 7-nitro-7-deazahypoxanthine (NitrocH) and 2-thiocytosine (sC), respectively, which are described in Lahoud et al. (2008) Nucleic Acids Research 36(22):6999-7008 (hereby incorporated by reference for this description). Hoshinka et al. (2010) Angew Chem Int Ed Engl. 49(32):5554-5557 describes the use of such bases (“Self-Avoiding Molecular Recognition Systems”), including 2′-hypoxanthine as G* (this reference is hereby incorporated by reference for this description; see especially, FIG. 1); see also Yang et al. (2015) Chembiochem. 16(9):1365-1367 (this reference is hereby incorporated by reference for this description; see especially, Scheme 1). The analogs tested in this study are shown in Formula 13.

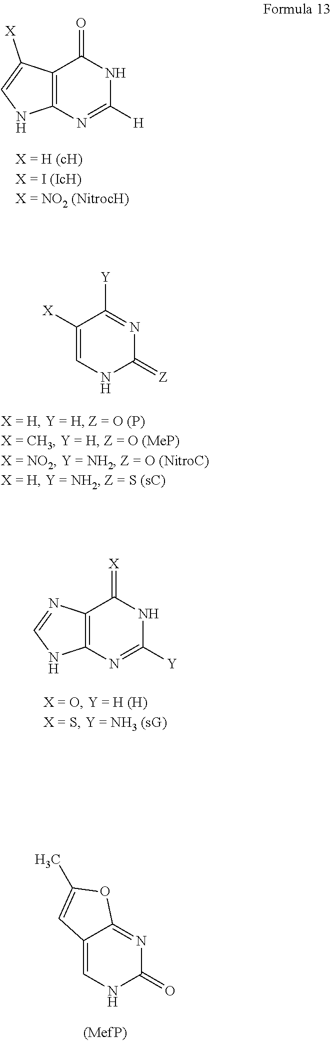

### Amplification

For amplification in any of the methods described herein, primers and any other appropriate oligonucleotides are contacted with sample nucleic acids under conditions wherein the primers anneal to their template strands, if present. In some embodiments, the amplification step is performed using PCR. Illustrative PCR reaction mixtures generally contain an appropriate buffer, a source of magnesium ions (Mg²⁺) in the range of about 1 to about 10 mM, e.g., in the range of about 2 to about 8 mM, nucleotides, and optionally, detergents, and stabilizers. An example of one suitable buffer is TRIS buffer at a concentration of about 5 mM to about 85 mM, with a concentration of 10 mM to 30 mM preferred. In one embodiment, the TRIS buffer concentration is 20 mM in the reaction mix double-strength (2X) form. The reaction mix can have a pH range of from about 7.5 to about 9,0, with a pH range of about 8.0 to about 8.5 as typical. Concentration of nucleotides can be in the range of about 25 mM to about 1000 mM, typically in the range of about 100 mM to about 800 mM. Examples of dNTP concentrations are 100, 200, 300, 400, 500, 600, 700, and 800 mM. Detergents such as Tween 20, Triton X 100, and Nonidet P40 may also be included in the reaction mixture. Stabilizing agents such as dithiothreitol (DTT, Cleland's reagent) or 2-mercaptoethanol may also be included. In addition, master mixes may optionally contain dUTP as well as uracil DNA glycosylase (uracil-N-glycosylase, UNG). A master mix is commercially available from Applied Biosystems, Foster City, Calif., (TaqMan® Universal Master Mix, cat. nos. 4304437, 4318157, and 4326708).

### Labeling Strategies

Any suitable labeling strategy can be employed in the methods described herein. Where the reaction is analyzed for presence of a single amplification product, a universal detection probe can be employed in the amplification mixture. In particular embodiments, real-time PCR detection can be carried out using a universal qPCR probe. Suitable universal qPCR probes include double-stranded DNA-binding dyes, such as SYBR Green, Pico Green (Molecular Probes, Inc., Eugene, Oreg.), Eva Green (Biotium), ethidium bromide, and the like (see Zhu et al., 1994, Anal. Chem. 66:1941-48).

In some embodiments, one or more target-specific qPCR probes (i.e., specific for a target nucleotide sequence to be detected) is employed in the amplification mixtures to detect amplification products. By judicious choice of labels, analyses can be conducted in which the different labels are excited and/or detected at different wavelengths in a single reaction (“multiplex detection”). See, e.g., Fluorescence Spectroscopy (Pence et al., Eds.) Marcel Dekker, New York, (1971); White et al., Fluorescence Analysis: A Practical Approach, Marcel Dekker, New York, (1970); Berlman, Handbook of Fluorescence Spectra of Aromatic Molecules, 2nd ed., Academic Press, New York, (1971); Griffiths, Colour and Constitution of Organic Molecules, Academic Press, New York, (1976); Indicators (Bishop, Ed.). Pergamon Press, Oxford, 19723; and Haugland, Handbook of Fluorescent Probes and Research Chemicals, Molecular Probes, Eugene (1992); and Linck et al. (2017) “A multiplex TaqMan qPCR assay for sensitive and rapid detection of phytoplasmas infecting Rubus species,” PLOS One 12(5).

In some embodiments, probes are designed so that annealing of the probe to a target nucleic acid leads to Fluorescence Resonance Energy Transfer (FRET). FRET is a quantum phenomenon occurring between two dye molecules. Excitation is transferred from a donor to an acceptor fluorophore, whereby the donor molecule fluorescence is quenched, and the acceptor molecule becomes excited. In certain embodiments, parts of a fluorophore-labeled DNA probe can participate in collisional and static fluorescence quenching. These non-FRET-based mechanisms can mimic the fluorescence quenching effects of FRET. The design of FRET and other fluorescence-based probes useful in real-time PCR reactions is well-known and reviewed, for example, in Didenko, Biotechniques (2001) 31:5, 1106-1121, which is incorporated by reference herein for this description.

In some embodiments, it may be convenient to include labels on one or more of the primers employed in in amplification mixture.

### Exemplary Automation and Systems

In some embodiments, a target nucleic acid is detected using an automated sample handling and/or analysis platform. In some embodiments, commercially available automated analysis platforms are utilized. For example, in some embodiments, the GeneXpert® system (Cepheid, Sunnyvale, Calif.) is utilized.

The methods described herein are illustrated for use with the GeneXpert system. Exemplary sample preparation and analysis methods are described below. However, the present invention is not limited to a particular detection method or analysis platform. One of skill in the art recognizes that any number of platforms and methods may be utilized.

The GeneXpert® utilizes a self-contained, single use cartridge. Sample extraction, amplification, and detection may all be carried out within this self-contained “laboratory in a cartridge” (available from Cepheid—see www.cepheid.com).

Components of the cartridge include, but are not limited to, processing chambers containing reagents, filters, and capture technologies useful to extract, purify, and amplify target nucleic acids. A valve enables fluid transfer from chamber to chamber and contains nucleic acids lysis and filtration components. An optical window enables real-time optical detection. A reaction tube enables very rapid thermal cycling.

In some embodiments, the GeneXpert® system includes a plurality of modules for scalability. Each module includes a plurality of cartridges, along with sample handling and analysis components.

After the sample is added to the cartridge, the sample is contacted with lysis buffer and released nucleic acid is bound to a nucleic acid-binding substrate such as a silica or glass substrate. The sample supernatant is then removed and the nucleic acid eluted in an elution buffer such as a Tris/EDTA buffer. The eluate may then be processed in the cartridge to detect target genes as described herein. In some embodiments, the eluate is used to reconstitute at least some of the reagents, which are present in the cartridge as lyophilized particles.

In some embodiments, PCR is used to amplify and detect the presence of one or more target nucleic acids. In some embodiments, the PCR uses Taq polymerase with hot start function, such as AptaTaq (Roche).

In some embodiments, an off-line centrifugation is used to improve assay results with samples with low cellular content. The sample, with or without the buffer added, is centrifuged and the supernatant removed. The pellet is then resuspended in a smaller volume of supernatant, buffer, or other liquid. The resuspended pellet is then added to a GeneXpert® cartridge as previously described.

### Kits

Also contemplated is a kit for carrying out the methods described herein. Such kits include one or more reagents useful for practicing any of these methods. A kit generally includes a package with one or more containers holding the reagents, as one or more separate compositions or, optionally, as an admixture where the compatibility of the reagents will allow. The kit can also include other material(s) that may be desirable from a user standpoint, such as a buffer(s), a diluent(s), a standard(s), and/or any other material useful in sample processing, washing, or conducting any other step of the assay.

Kits preferably include instructions for carrying out one or more of the screening methods described herein. Instructions included in kits can be affixed to packaging material or can be included as a package insert. While the instructions are typically written or printed materials, they are not limited to such. Any medium capable of storing such instructions and communicating them to an end user can be employed. Such media include, hut are not limited to, electronic storage media (e.g., magnetic discs, tapes, cartridges, chips), optical media (e.g., CD ROM), and the like. As used herein, the term “instructions” can include the address of an internet site that provides the instructions.

### Examples

### Example 1: Pseudo-Complementary Blocker Oligonucleotide does not Interfere with Sequence-Specific Probe Capture by Pseudo-Complementary Capture Oligonucleotide

A liquid phase hybridization was carried out using the oligonucleotides shown in Table 1 below to test the effect of including a pseudo-complementary oligonucleotide blocker on sequence specific capture by a pseudo-complementary capture oligonucleotide.

TABLE 1
Oligonucleotides

| SEQ | | | | Oligo | |
|---|---|---|---|---|---|
| ID | | Oligo | | Sequence* | |
| NO: | Function | Name | 5-Mod | (5′-3′) | 3-Mod |
| 1 | | | | | |
| 2 | Fluorescent, | KRAS | Biotin | TGGAGC | FAM |
| | mutation- | Mut | | (2thioT) | |
| | specific, | 2thioT | | TG(2thioT) | |
| | biotinylated | Biotin- | | GGCG | |
| | and | FAM | | (2thioT) | |
| | pseudo- | | | AGG | |
| | complementary | | | | |
| | capture oligo | | | | |
| 3 | Quenching | KRAS | | C(sT) | CDQ77 |
| | complement | Mut | | A(sC) | |
| | to | CDQ77 | | | |
| | capture | 3′ | | GACACCA | |
| | oligo, 3′ | | | GCTCC | |
| | quencher | | | | |
| 4 | Quenching | KRAS | CDQ77 | C(sT)A(sC) | |
| | complement to | Mut | | GACACCAGC | |
| | capture oligo, | CDQ77 | | TCC | |
| | 5′ quencher | 5′ | | | |
| 5 | Pseudo- | KRAS | | C(sT) | |
| | complementary, | WT A01 | | (A01)(sC) | |
| | wild-type | | | GCC(A01) | |
| | blocker | | | CC(A01) | |
| | | | | GCTCC | |

2thioT = 2-thiothymine, A01 = 2-aminoadenine (aka diaminopurine), FAM = fluorescein, sT = stabilized deoxythymidine, sC = stabilized deoxycytidine, CDQ77 = quencher

First, 300 nM of the Fluorescent, mutation-specific biotinylated and pseudo-complementary capture oligo was incubated with either the Quenching complement oligo (3′ quencher) or the Quenching complement oligo (5′ quencher); both of these quenchers were fully complementary to the mutation-specific capture oligonucleotide. The concentration of the quenching complement in the reactions were 3000 nM, 300 nM, 30 nM, or 0 nM, The oligos were combined in a final reaction volume of 10 μl a 10 mM Tris-HCL, 0.1 mM EDTA, 8.0 buffer and pipetted into a 384-well plate.

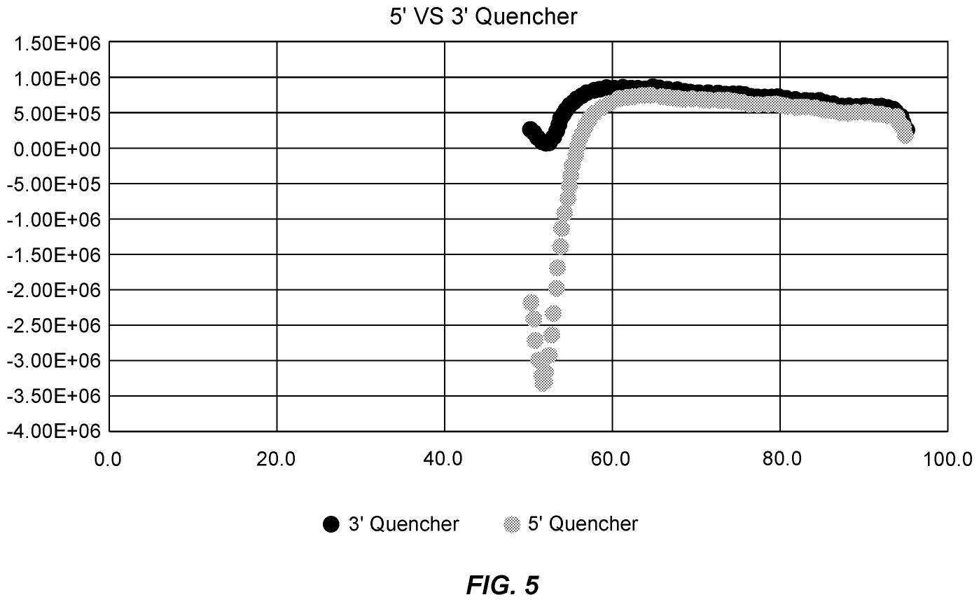

The oligo reaction volumes were subjected to a melt curve analysis in a QUANTSTUDIO 7. The melt curve protocol was 95° C. for 15 sec, cooled to 40° C. at a ramp rate of 1.6° C./sec, and heated to 95° C. at a ramp rate of 0.05° C./sec. The results, shown in FIG. 5, established that the oligo with the 5′ quencher performed better than the oligo with the 3′ quencher.

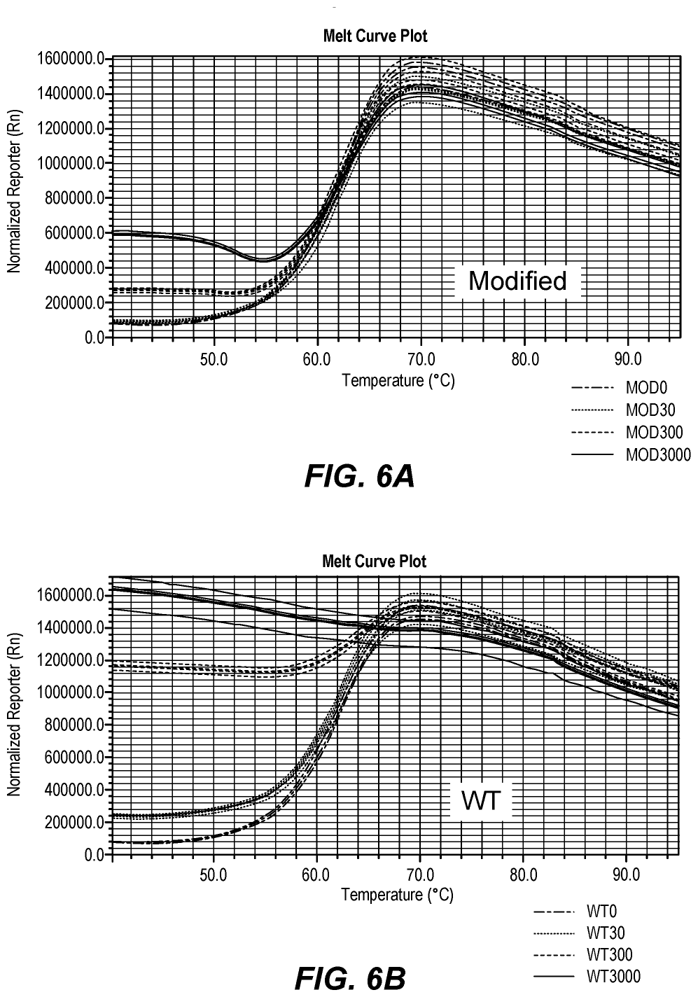

Next, the 5′ quencher was used in assays to determine the effect of including either a Pseudo-complementary, wild-type blocker or a Complementary, wild-type blocker (i.e., one that contained no pseudo-complementary bases. 10 μl reaction volumes were made containing 50 mM KCl, 2.5 mM MgCl₂, 0.2 mM dNTPs, and 10 U of CAT-A enzyme. Each reaction volume contained a mixture of oligos including 300 nM Capture oligo and 300 Quencher oligo (5′ quencher). The experimental conditions were: the addition of 3000 nM, 300 nM, 30 nM, or 0 nM of either the Complementary, wild-type blocker or the Pseudo-complementary, wild-type blocker. The oligo volumes were subjected to a melt curve analysis in a QUANTSTUDIO 7. The melt curve protocol was 95° C. for 15 sec, cooled to 40° C. at a ramp rate of 1.6° C./sec, and heated to 95° C. at a ramp rate of 0.05° C./sec. 6 replicates per condition were run. The results, shown in FIGS. 6A-6B show that the Pseudo-complementary, wild-type blocker does not interfere with hybridization of the Quencher oligo to the pseudo-complementary capture oligo, whereas the Complementary, wild-type blocker does.

### REFERENCES

1. Kutyavin et al., U.S. Pat. No. 5,912,340, issued Jun. 15, 1999);
2. Woo et al. (1996) Nucleic Acids Research 24(13):2470-2475;
3. Lahoud et al. (2008) Nucleic Acids Research 36(10):3409-3419);
4. Benner et al., U.S. Pat. No. 8,871,469, issued Oct. 28, 2014;
5. Lahoud et al, (2008) Nucleic Acids Research 36(22):6999-7008;
6. Hoshika et al. (2010) Angew Chem Int Ed Engl. 49(32):5554-5557;
7. Yang (2015) Chembiochem. 16(9):1365.1370;
8. Dominguez and Kolodney (2005) 24:6830-6834;
9. Albitar, U.S. Pat. No. 10,227,657, issued Mar. 12, 2019;
10. Didenko, Biotechniques (2001) 31:5, 1106-1121.

What is claimed is:

1. A method of determining whether a nucleotide sequence is present in a target nucleic acid sequence in a sample, wherein the target nucleic acid sequence comprises a polymorphic site, wherein the polymorphic site is characterized by a first nucleotide sequence and a second nucleotide sequence, wherein the first and second nucleotide sequences differ by at least one nucleotide or ribonucleotide, the method comprising:
contacting nucleic acid of, or derived from, the sample with forward and reverse primers capable of amplifying the target nucleic acid sequence, wherein said contacting is in the presence of a blocker oligonucleotide that is complementary to the first nucleotide sequence to form a reaction mixture, wherein:
if the target nucleic acid sequence comprises the first nucleotide sequence, the blocker oligonucleotide anneals to the first nucleotide sequence and inhibits amplification; or
if the target nucleic acid sequence comprises the second nucleotide sequence, the blocker oligonucleotide does not anneal to the second nucleotide sequence and does not inhibit amplification;
conducting an amplification reaction in the reaction mixture;
after the amplification reaction, contacting the reaction mixture, or nucleic acids from the reaction mixture, with a capture oligonucleotide that is complementary to the second nucleotide sequence under conditions suitable for specific hybridization; and
detecting any specific hybridization to the capture oligonucleotide, wherein the presence of specific hybridization to the capture oligonucleotide indicates that the second nucleotide sequence is present in the target nucleic acid sequence,
wherein:
the blocker oligonucleotide comprises one or more first modified bases and the capture oligonucleotide comprises one or more second modified bases, at least one of which is complementary to one of the first modified bases, wherein the modified bases preferentially pair with unmodified forms of their complementary bases, as compared to pairing between modified, complementary bases; and
the presence of the one or more modified bases in the blocker oligonucleotide and in the capture oligonucleotide destabilizes hybridization between the blocker oligonucleotide and the capture oligonucleotide.

2. The method of claim 1, wherein at least one of the first modified bases and at least one of the second, complementary modified bases in the capture oligonucleotide are bases that do not differ between the first and second nucleotide sequence.

3. The method of claim 1 or claim 2, wherein the method comprises quantifying any specific hybridization to the capture oligonucleotide.

4. The method of any one of claims 1-3, wherein the sample consists of nucleic acids from a single cell.

5. An oligonucleotide set comprising:
forward and reverse primers capable of amplifying a target nucleic acid sequence, wherein the target nucleic acid sequence comprises a polymorphic site, wherein the polymorphic site is characterized by a first nucleotide sequence and a second nucleotide sequence, wherein the first and second nucleotide sequences differ by at least one nucleotide or ribonucleotide;
a blocker oligonucleotide that is complementary to the first nucleotide sequence; and
a capture oligonucleotide that is complementary to the second nucleotide sequence,
wherein the blocker oligonucleotide comprises one or more first modified bases and the capture oligonucleotide comprises one or more second modified bases, at least one of which is complementary to one of the first modified bases, wherein the modified bases preferentially pair with unmodified forms of their complementary bases, as compared to pairing between modified, complementary bases; and
the presence of the one or more modified bases in the blocker oligonucleotide and in the capture oligonucleotide destabilizes hybridization between the blocker oligonucleotide and the capture oligonucleotide.

6. The method or oligonucleotide set of any one of the preceding claims, wherein the capture oligonucleotide is attached to a support.

7. A method of simplifying preparations for nucleic acid sequencing, the method comprising:
adding DNA sequencing adaptors to nucleic acid fragments to produce sequencing templates;
amplifying sequencing templates to produce amplified DNA templates; and
contacting the amplified DNA templates with capture oligonucleotides attached to a support under conditions suitable for hybridization, wherein the DNA sequencing adaptors and the capture oligonucleotides comprise complementary nucleotide sequences,
wherein:
the DNA sequencing adaptors each comprise one or more first modified bases in their complementary nucleotide sequence, and the capture oligonucleotides comprise one or more second modified bases in their complementary nucleotide sequence, wherein at least one of the first and second modified bases are complementary, wherein the modified bases preferentially pair with unmodified forms of their complementary bases, as compared to pairing between modified, complementary bases; and
hybridization of amplified DNA templates to the capture oligonucleotide is favored over hybridization of free adaptors to the capture oligonucleotides, eliminating a need to separate amplified DNA templates from free adaptors before further DNA sequencing steps.

8. A combination of components for simplifying nucleic acid sequencing, the combination comprising:
DNA sequencing adaptors; and
capture oligonucleotides attached to, or adapted to be attached to, a support, wherein the DNA sequencing adaptors and the capture oligonucleotides comprise complementary nucleotide sequences,
wherein:
the DNA sequencing adaptors each comprise one or more first modified bases in their complementary nucleotide sequence, and the capture oligonucleotides comprise one or more second modified bases in their complementary nucleotide sequence, wherein at least one of the first and second modified bases are complementary, wherein the modified bases preferentially pair with unmodified forms of their complementary bases, as compared to pairing between modified, complementary bases; and
hybridization of amplified DNA templates to the capture oligonucleotide is favored over hybridization of free adaptors to the capture oligonucleotides, eliminating a need to separate amplified DNA templates from free adaptors.

9. The method of claim 7 or the combination of components of claim 8, wherein the DNA sequencing adaptors comprise a nucleotide sequence that is a binding site for a DNA sequencing primer and a barcode nucleotide sequence.

10. The method of claim 7, wherein the method additionally comprises producing the nucleic acid fragments from genomic DNA, or the combination of components of claim 8, wherein the combination additionally comprises one or more reagents that produce the nucleic acid fragments from genomic DNA.

11. The method of claim 7, wherein said adding of DNA sequencing adaptors comprises ligating the DNA sequencing adaptors to the nucleic acid fragments, or the combination of components of claim 8, wherein the combination additionally comprises a ligase.

12. The method or combination of components of any one of claims 7-11, wherein the method employs, or the combination comprises, a DNA polymerase for amplification.

13. The method or combination of components of any one of claims 7-12, wherein the method employs, or the combination comprises, a reverse transcriptase for reverse-transcribing nucleic acid fragment that are RNA.

14. The method, oligonucleotide set, or combination of components of any one of the preceding claims, wherein modified complementary bases form fewer hydrogen bonds with each other than with unmodified complementary bases.

15. The method, oligonucleotide set, or combination of components of claim 14, wherein the Li of a base pair formed between modified complementary bases less than 40° C.

16. The method, oligonucleotide set, or combination of components of any one of the preceding claims, wherein at least one complementary pair of modified bases comprises modified forms of adenine and thymine.

17. The method, oligonucleotide set, or combination of components of any one of the preceding claims, wherein at least one complementary pair of modified bases comprises modified forms of guanine and cytosine.

18. The method, oligonucleotide set, or combination of components of any one of the preceding claims, wherein the blocker oligonucleotide is blocked to 3′ extension.
